(19) United States
(12) Patent Application Publication
(10) Pub. No.: US 2019/0203364 A1
(43) Pub. Date: Jul. 4, 2019

(54) APPARATUS AND METHOD FOR ELECTROCHEMICAL REDUCTION OF BIOCHEMICAL COMPOSITIONS FOR BIOCONJUGATION

(71) Applicant: Ligare Biotechnologies, Inc., Phoenix, AZ (US)

(72) Inventor: Derrick Houser, San Francisco, CA (US)

(21) Appl. No.: 16/099,374

(22) PCT Filed: May 5, 2017
(86) PCT No.: PCT/US2017/031364

§ 371 Date: Nov. 6, 2018

Related U.S. Application Data
(60) Provisional application No. 62/332,716, filed on May 6, 2016.

(51) Int. Cl. C25B 3/04; C25B 15/02; C25B 9/08; C25B 13/08; C25B 11/04; C25B 11/12; C07K 1/113

(52) U.S. Cl. CPC C25B 3/04; C25B 15/02; C25B 9/08; C07K 1/1133; C25B 11/0473; C25B 11/12; C25B 13/08

(57) ABSTRACT

Disclosed herein are methods and devices for performing electrochemical reduction of disulfide and related bonds in biochemical compositions such as proteins for improved bioconjugation reactions.

### CROSS-REFERENCE

This application claims the benefit of U.S. Provisional Application No. 62/332,716, filed on May 6, 2016, which application is incorporated herein by reference.

### BACKGROUND

Bioconjugation involves forming a stable covalent bond between two molecules, at least one of which is a biomolecule. Bioconjugation can be accomplished in part by a chemical reaction of a linker with a chemically active site on the biomolecule. One such chemically active site is the sulfhydryl group (—SH), which is present in the amino acid residue cysteine. Sulfhydryl groups in biomolecules are generally masked as disulfide bonds (—S—S—) between spatially adjacent cysteine residues. These disulfide bonds must be reduced to create chemical linkers such as sulfhydryl groups. Historically, reduction of disulfide bonds in biomolecules has been done with chemical reducing agents such as dithiothreitol (“DTT”) and tris(2-carboxyethyl)phosphine (“TCEP”). Drawbacks of using such chemical reducing agents can include long reaction times (e.g., up to 24 hours) and difficulty purifying the biomolecules to remove the chemical reducing agents or oxidized forms thereof for subsequent bioconjugation. Provided herein are apparatuses and methods that address the foregoing.

### SUMMARY

Disclosed herein, in certain embodiments, are electrochemical devices comprising a first chamber comprising an inserted first electrode, a second chamber comprising an inserted second electrode, and a porous membrane which separates the first and second chamber and is configured to provide electrochemical communication between the first chamber and the second chamber, wherein the first and second electrodes are configured to work together to electrochemically reduce one or more biochemical compositions placed in the first chamber when voltage is applied across the first and second electrodes. In some embodiments, the volume of the second chamber is larger than the volume of the first chamber. In some embodiments, the first chamber is contained within the second chamber. In some embodiments, the volume of the second chamber is at least 5×, at least 10×, at least 20×, at least 35×, at least 50×, at least 100×, at least 250×, at least 500×, or at least 1000× larger than the volume of the first chamber. In some embodiments, the volume of the first chamber is about 3 mL and the volume of the second chamber is about 50 mL. In some embodiments, the volume of the first chamber is about 300 μL, and the volume of the second chamber is at least 150 mL. In some embodiments, the first chamber is comprised of a well in a 96-well plate, and the second chamber is a rectangular chamber attached to the bottom of the 96-well plate, wherein the second chamber is in electrochemical communication with each well in the 96-well plate by means of a porous membrane that separates each well from the rectangular chamber. In some embodiments, the device is configured to provide a first electrode to two or more wells of the 96-well plate, and a single second electrode to the rectangular chamber, such that two or more samples of a biochemical composition may be reduced in parallel. In some embodiments, the two or more samples comprise different biochemical compositions. In some embodiments, the first chamber contains a magnetic stir bar configured to agitate the one or more biochemical compositions. In some embodiments, the porous membrane is a cellulose membrane. In some embodiments, the porous membrane comprises a pore size small enough to retain the biochemical composition. In some embodiments, the biochemical composition is a protein of molecular weight about 150 kDa. In some embodiments, the biochemical composition is a protein of molecular weight about 50 kDa. In some embodiments, the first electrode is a negative electrode (or cathode). In some embodiments, the first electrode is a working electrode. In some embodiments, the negative electrode comprises platinum. In some embodiments, the first electrode does not comprise titanium. In some embodiments, the working electrode is connected to a direct current power supply. In some embodiments, the second electrode is a positive electrode (or anode). In some embodiments, the second electrode is a counter electrode. In some embodiments, the positive electrode comprises carbon. In some embodiments, the biochemical composition is a protein. In some embodiments, the protein is an antibody or an antibody fragment. In some embodiments, the first chamber and the second chamber comprise a buffer. In some embodiments, the buffer comprises PBS. In some embodiments, the buffer comprises glucose and EDTA. In some embodiments, the first electrode and second electrode are submerged in the buffer. In some embodiments, the first and second electrodes are configured to work together to electrochemically reduce disulfide, sulfur-selenium bonds, diselenide bonds, or a combination thereof, of the biochemical composition.

Disclosed herein, in certain embodiments, are methods for reducing disulfide bonds, sulfur-selenium bonds, or diselenide bonds in a biochemical composition comprising inserting the biochemical composition into an electrochemical cell comprising a first chamber comprising an inserted first electrode, a second chamber comprising an inserted second electrode, and a porous membrane which separates the first and second chamber and is configured to provide electrochemical communication between the first chamber and the second chamber, agitating the biochemical composition with a magnetic stir bar, applying a voltage across the first and second electrodes of the electrochemical cell, thereby reducing the disulfide bonds, sulfur-selenium bonds, or diselenide bonds of the biochemical composition. In some embodiments, the voltage is 3 V. In some embodiments, the voltage is 1.5 V. In some embodiments, the volume of the second chamber is larger than the volume of the first chamber. In some embodiments, the first chamber is contained within the second chamber. In some embodiments, the volume of the second chamber is at least 5×, at least 10×, at least 20×, at least 35×, at least 50×, at least 100×, at least 250×, at least 500×, or at least 1000× larger than the volume of the first chamber. In some embodiments, the volume of the first chamber is about 3 mL and the volume of the second chamber is about 50 mL. In some embodiments, the volume of the first chamber is about 300 μL, and the volume of the second chamber is at least 150 mL. In some embodiments, the first chamber is comprised of a well in a 96-well plate, and the second chamber is a rectangular chamber attached to the bottom of the 96-well plate, wherein the second chamber is in electrochemical communication with each well in the 96-well plate by means of a porous membrane that separates each well from the rectangular chamber. In some embodiments, the electrochemical cell is configured to provide a first electrode to two or more wells of the 96-well plate, and a single second electrode to the rectangular chamber, such that two or more samples of a biochemical composition may be reduced in parallel. In some embodiments, the two or more samples comprise different biochemical compositions. In some embodiments, the first chamber contains the magnetic stir bar configured to agitate the one or more biochemical compositions. In some embodiments, the porous membrane is a cellulose membrane. In some embodiments, the porous membrane comprises a pore size small enough to retain the biochemical composition. In some embodiments, the biochemical composition is a protein of molecular weight about 150 kDa. In some embodiments, the biochemical composition is a protein of molecular weight about 50 kDa. In some embodiments, the first electrode is a negative electrode (or cathode). In some embodiments, the first electrode is a working electrode. In some embodiments, the negative electrode comprises platinum. In some embodiments, the first electrode does not comprise titanium. In some embodiments, the working electrode is connected to a direct current power supply. In some embodiments, the second electrode is a positive electrode (or anode). In some embodiments, the second electrode is a counter electrode. In some embodiments, the positive electrode comprises carbon. In some embodiments, the biochemical composition is a protein. In some embodiments, the protein is an antibody or an antibody fragment. In some embodiments, the first chamber and the second chamber comprise a buffer. In some embodiments, the buffer comprises PBS. In some embodiments, the buffer comprises glucose and EDTA. In some embodiments, the first electrode and second electrode are submerged in the buffer. In some embodiments, the voltage or a process time are adjusted to control the extent of the reduction of the disulfide bonds, sulfur-selenium bonds, or diselenide bonds of the biochemical composition. In some embodiments, the method further comprises conjugating a drug molecule, a detection agent, an imaging agent, a peptide, a protein, or an oligonucleotide to one or more sulfydryl groups derived from the disulfide bonds in a conjugation step. In some embodiments, the conjugation step is performed in a buffer previously used for the reduction step without changing the buffer.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A provides a schematic that illustrates chemical reduction of disulfide bonds in a biomolecule and conjugation of drug to resulting sulfhydryl groups.

FIG. 1B provides a schematic that illustrates electrochemical reduction of disulfide bonds in a biomolecule and conjugation of a drug to resulting sulfhydryl groups in accordance with some embodiments.

FIG. 2A provides a schematic that illustrates an apparatus for electrochemical reduction in accordance with some embodiments.

FIG. 2B provides an image of an apparatus for electrochemical reduction in accordance with some embodiments.

FIG. 3 provides a schematic that illustrates an example electrochemical reduction of disulfide bonds in a F(ab′)₂ fragment of an antibody in accordance with some embodiments.

FIG. 4A provides an image of a protein electrophoretogram for an example electrochemical reduction of disulfide bonds in a F(ab′)₂ fragment of an antibody at 3 V over various time periods in accordance with some embodiments.

FIG. 4B provides an image of an electrophoretogram for an example electrochemical reduction of disulfide bonds in a F(ab′)₂ fragment of an antibody at 1.5 V over various time periods in accordance with some embodiments.

FIG. 5A provides an illustration of one non-limiting example of an electrochemical device comprising a 3 mL reaction chamber and a 50 mL exterior chamber fabricated from a conical centrifugation tube.

FIG. 5B shows a photograph of an electrochemical device comprising a plurality of 300 reaction chambers fashioned from the wells of a 96-well plate, where a working electrode is inserted into one or more of the wells. The bottom of the wells have been removed and a dialysis membrane is affixed to the bottom of each well. The counter electrode is located in a rectangular bottom chamber fixed to the underside of the 96-well plate. In some embodiments, a single working electrode is inserted into a single well. In some embodiments, two or more working electrodes, e.g., up to 96 working electrodes, are inserted into a corresponding number of wells to perform reduction of disulfide or related bonds in a plurality of biochemical composition samples.

FIG. 6 shows an electrophoretogram of a mIgG2a sample reduced at 1.5 V (voltage B) for twenty minutes and a mIgG2a antibody reduced at 3 V (voltage A) for twenty minutes.

FIG. 7 shows data for the free sulfhydryl concentration for samples reduced under voltage A (3 V), voltage B (1.5 V), and voltage C (constant current of 2 mA, dynamic voltage between 1.5-2.5 V) conditions as a function of time. Inset: standard curve used for quantitation of free sulfhydryl groups using DTNB (Ellman's reagent).

FIG. 8A shows an HPLC chromatograph of fluorophore conjugated mIgG2a, after reduction at 1.5 V of electrical potential, as a function of time.

FIG. 8B shows an HPLC chromatograph of total mIgG2a concentration, after reduction at 1.5 V of electrical potential, as a function of time.

FIG. 9A an HPLC chromatograph for the electrochemically reduced sample with both a fluorescence trace to detect fluorophore concentration and an absorbance trace to detect antibody concentration. The inset in the graph depicts an antibody conjugated to a maleimide-CY5 fluorophore.

FIG. 9B shows an HPLC chromatograph for the chemically reduced sample with both a fluorescence trace and an absorbance trace.

### DETAILED DESCRIPTION

Provided herein, in some embodiments, is an electrochemical apparatus including a first chamber configured to agitate an electrolyte including a biochemical composition; a second, larger chamber; a porous membrane separating the first chamber from the second chamber; a first “working” electrode inserted in the first chamber; and a second, larger “counter” electrode inserted in the second chamber. The porous membrane can be configured to provide electrochemical communication between the first and second chambers when the apparatus is filled with an electrolyte. In some embodiments, the porous membrane is a regenerated cellulose membrane with pores sized to retain a biochemical composition including at least a 150 kDa protein. The first and second electrodes can be configured to work together to electrochemically reduce one or more residues of a biochemical composition when a voltage is applied across the first and second electrodes.

Also provided herein, in some embodiments, is an electrochemical reduction method comprising loading a biochemical composition into a first chamber including a first electrode; agitating the biochemical composition; and applying a voltage across the first electrode and a second electrode for a period of time; and reducing disulfide bonds, sulfur-selenium bonds, diselenide bonds, or a combination thereof in the biochemical composition in a reduction step. The first electrode can be in electrochemical communication with the second electrode in a second chamber across a porous membrane separating the first chamber from the second chamber. In some embodiments, the method further comprises conjugating a drug or chemical moiety to one or more sulfhydryl groups derived from the disulfide bonds in a conjugation step, wherein the conjugation step is performed in a buffer previously used for the reduction step without changing out the buffer.

These and other features of the concepts provided herein may be better understood with reference to the following drawings, description, and appended claims.

Before certain concepts and some embodiments thereof are provided in greater detail, it should be understood by persons of ordinary skill in the art that the concepts and embodiments provided herein are not limiting. For example, it should be understood that one or more elements in any embodiment provided herein can vary. In view of the foregoing, one or more elements from one or more embodiments can be combined with elements of any other embodiments, substituted for elements of any other embodiments, or some combination thereof.

It should also be understood that the terminology used herein is for the purpose of describing the concepts and embodiments provided herein, and the terminology is not intended to be limiting. Unless indicated otherwise, ordinal numbers (e.g., first, second, third, etc.) are used to distinguish or identify different elements or steps respectively in a group of elements or group of steps. The ordinal numbers do not supply a serial or numerical limitation. For example, “first,” “second,” and “third” elements or steps need not necessarily appear in that order, and the embodiments need not necessarily be limited to the three elements or steps. Unless indicated otherwise, labels such as “left,” “right,” “front,” “back,” “top,” “bottom,” “forward,” “reverse,” “clockwise,” “counter clockwise,” “up,” “down,” or other similar terms such as “upper,” “lower,” “aft,” “fore,” “vertical,” “horizontal,” “proximal,” “distal,” and the like are not intended to imply, for example, any particular fixed location, orientation, or direction. Instead, such labels are used to reflect, for example, relative location, orientation, or directions. It should also be understood that the singular forms of “a,” “an,” and “the” include plural references unless the context clearly dictates otherwise.

Unless defined otherwise, all technical and scientific terms used herein have the same meaning as commonly understood by persons of ordinary skill in the art.

Biochemical Compositions:

As used herein, the phrase “biochemical composition” (also referred to as a “biomolecule”) may refer to any of a variety of biological molecules known to those of skill in the art including, but not limited to, peptides, proteins, antibodies, enzymes, receptors, or fragments thereof. In some preferred embodiments, the disclosed methods and apparatus may be implemented to perform electrochemical reduction of antibodies and antibody fragments.

Chemical Bonds:

The methods and apparatus of the present disclosure may be utilized for electrochemical reduction of any chemical bond that is susceptible to reduction by chemical means. Examples include, but are not limited to, disulfide bonds, sulfur-selenium bonds, diselenide bonds, carbon-oxygen bonds, carbon-carbon bonds, carbon-sulfur bonds and carbon-phosphorus bonds as well as reduction of allylic and benzylic halides, or a combination thereof. In some preferred embodiments, the disclosed methods and apparatus are utilized for electrochemical reduction of disulfide bonds in protein molecules, e.g., antibody molecules or fragments thereof.

Bioconjugation:

Bioconjugation involves forming a stable covalent bond between two molecules, at least one of which is a biomolecule. Bioconjugation can be accomplished in part by a chemical reaction of a chemical moiety or linker molecule with a chemically active site on the biomolecule. One such chemically active site is the sulfhydryl group (—SH), which is present in the amino acid residue cysteine. Sulfhydryl groups in biomolecules are generally masked as disulfide bonds (—S—S—) between spatially adjacent cysteine residues. These disulfide bonds must be reduced to create chemically reactive groups such as sulfhydryl groups. Historically, reduction of disulfide bonds in biomolecules has been done with chemical reducing agents such as dithiothreitol (“DTT”) and tris(2-carboxyethyl)phosphine (“TCEP”). Drawbacks of using such chemical reducing agents can include long reaction times (e.g., up to 24 hours) and difficulty purifying the biomolecules to remove the chemical reducing agents or oxidized forms thereof for subsequent bioconjugation. Provided herein are apparatuses and methods that address the foregoing.

Any of a variety of sulfhydryl-reactive (or thiol-reactive) conjugation chemistries may be used to couple chemical moieties to sulfhydryl groups. Examples include, but are not limited to, use of haloacetyls, maleimides, aziridines, acryloyls, arylating agents, vinylsulfones, pyridyl disulfides, TNB-thiols and other sulfhydryl-reactive or thiol-reactive agents. Many of these groups conjugate to sulfhydryl groups through either alkylation (e.g., by formation of a thioether bond) or disulfide exchange (e.g., by formation of a disulfide bond).

Any of a variety of chemical moieties may be conjugated to sulfhydryl groups depending on the intended application. Examples include, but are not limited to, small organic molecules, drug molecules, linker molecules, radioactive labels or tags, fluorescent labels or tags, other detection or imaging agents, nanoparticles, colloidal particles, magnetic beads, protein molecules (e.g., antibodies, enzymes, or receptors) or fragments thereof, and oligonucleotides or nucleic acid molecules selected from the group consisting of: a morpholino, a peptide nucleic acid (PNA), a thioester peptide nucleic acid (tPNA), a locked nucleic acid (LNA), a phosphorothioate, a phosphonoacetate (PACE) phosphoramidite, a ribonucleic acid (RNA), and a deoxyribonucleic acid (DNA), etc.

Applications:

Bioconjugates prepared using the disclosed methods and apparatus may be used for any of a variety of applications known to those of skill in the art. Examples include, but are not limited to, drug delivery, in vitro or in vivo imaging, separation and purification processes, quantitative or qualitative assessment of biomolecules and the like. Uses of electrochemistry in proteomics include, but are not limited to oxidation or reduction of peptides, peptide bond cleavage, disulfide reduction, desalting proteins, protein oxidation for surface mapping, oxidation of DNA, nucleosides, etc., and the like.

Methods and Apparatus:

As noted above, in some embodiments, an apparatus is provided including a first chamber configured to agitate an electrolyte including a biochemical composition; a second, larger chamber; a porous membrane separating the first chamber from the second chamber; a first electrode inserted in the first chamber; and a second, larger electrode inserted in the second chamber. The porous membrane can be configured to provide electrochemical communication between the first and second chambers when the apparatus is filled with an electrolyte. In some embodiments, the porous membrane is a regenerated cellulose membrane with pores sized to retain a biochemical composition including at least a 150 kDa protein. The first and second electrodes can be configured to work together to electrochemically reduce one or more residues of a biochemical composition when a voltage is applied across the first and second electrodes.

As noted above, in some embodiments, a method is provided including loading a biochemical composition into a first chamber including a first electrode; agitating the biochemical composition; and applying a voltage across the first electrode and a second electrode for a period of time; and reducing disulfide bonds, sulfur-selenium bonds, diselenide bonds, or a combination thereof in the biochemical composition in a reduction step. The first electrode can be in electrochemical communication with the second electrode in a second chamber across a porous membrane separating the first chamber from the second chamber. In some embodiments, the method further comprises conjugating a drug or chemical moiety to one or more sulfhydryl groups derived from the disulfide bonds in a conjugation step, wherein the conjugation step is performed in a buffer previously used for the reduction step without changing out the buffer.

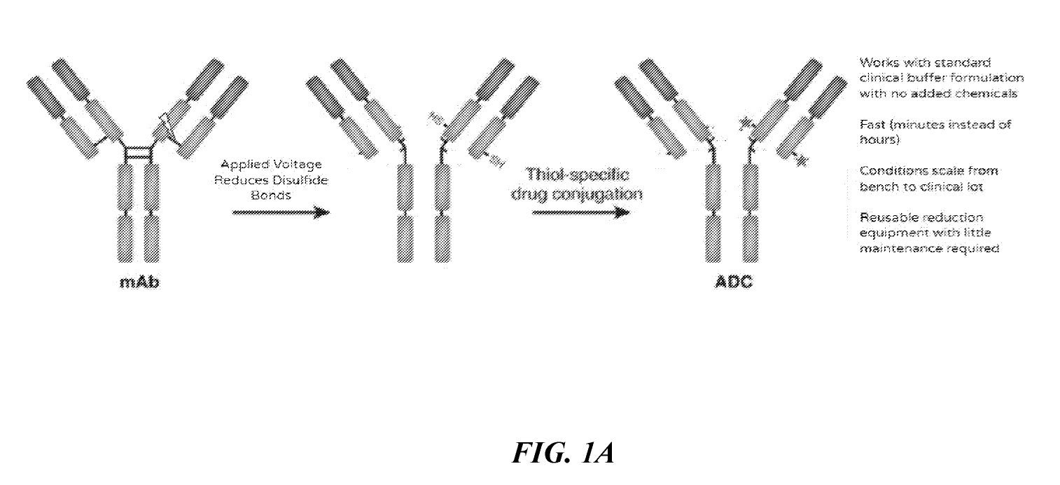

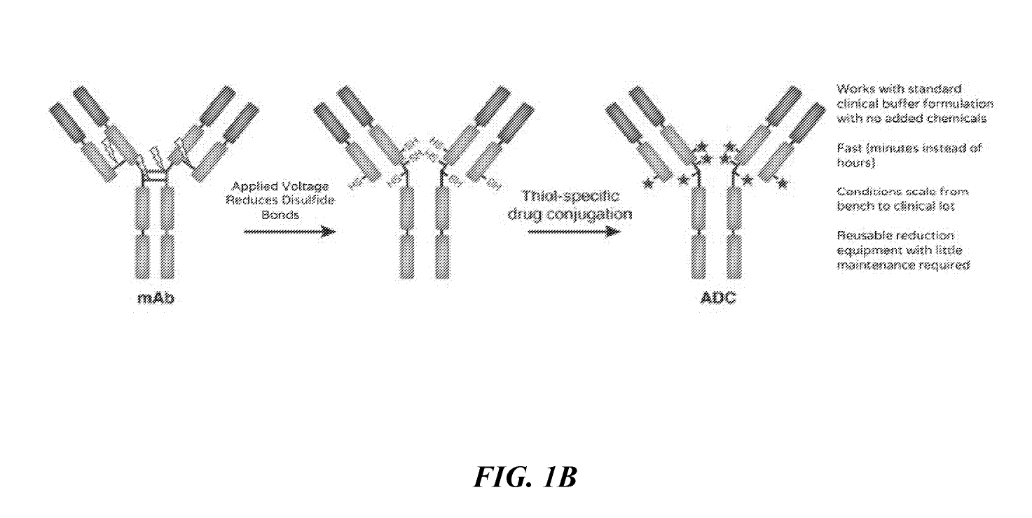

FIG. 1A provides a schematic that illustrates chemical reduction of disulfide bonds in a biomolecule and conjugation of a drug to resulting sulfhydryl groups.

As shown, DTT or TCEP can be used to chemically reduce disulfide bonds in a monoclonal antibody (“mAb”) to yield sulfhydryl groups, to one or more of which a drug or chemical moiety can be subsequently conjugated. Drawbacks of chemical reduction of disulfide bonds include costly chemical reducing agents, slow reaction rates, unknown scalability, and extensive purification to remove excess chemical reducing agents and oxidized forms thereof.

FIG. 1B provides a schematic that illustrates electrochemical reduction of disulfide bonds in a biomolecule and conjugation of drug to resulting sulfhydryl groups in accordance with some embodiments.

As shown, and in accordance with the concepts provided herein, disulfide bonds can be electrochemically reduced in a mAb (or other biomolecule) to yield sulfhydryl groups, to one or more of which a drug (e.g., chemotherapy agent) can be subsequently conjugated to yield an ADC. In some embodiments, other chemical moieties, e.g., radioactive labels or tags, fluorescent labels of tags, nanoparticles, colloidal particles, magnetic beads, etc., may be conjugated to the free sulfhydryl groups using any of a variety of sulfydryl-reactive conjugation chemistries known to those of skill in the art. Such conjugates may be used in a variety of applications including, but not limited to, drug delivery, in vitro or in vivo imaging, separation and purification processes, quantitative or qualitative assessment of biomolecules and the like. Benefits of electrochemical reduction of disulfide bonds include compatibility with clinical buffer formulations, fast reaction rates, scalability, and reusability of electrochemical reduction equipment.

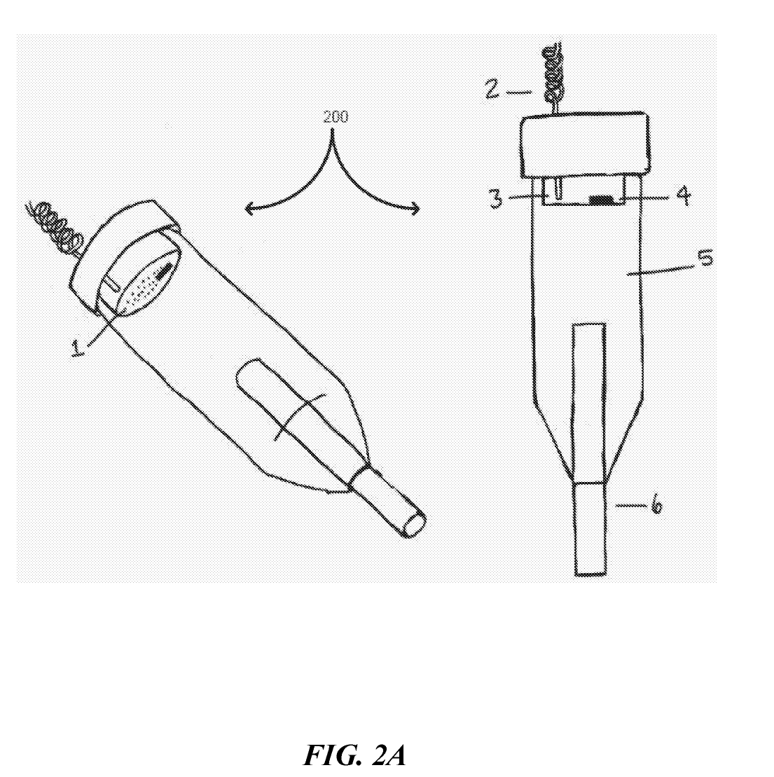

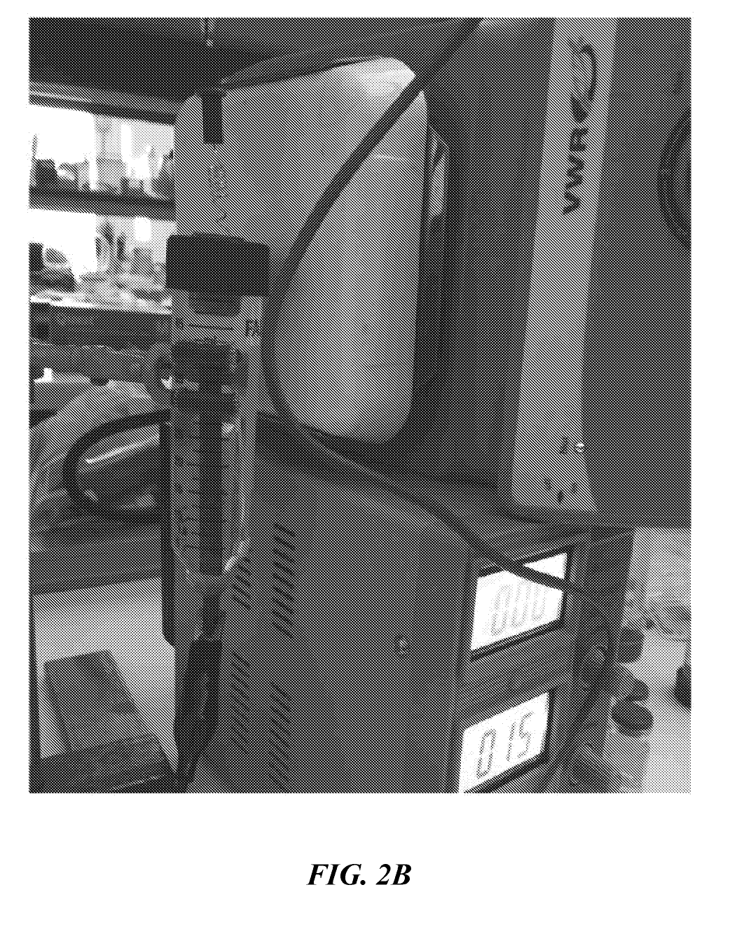

FIG. 2A provides a schematic that illustrates an apparatus 200 for electrochemical reduction in accordance with some embodiments. FIG. 2B provides an image of the apparatus 200 for electrochemical reduction in accordance with some embodiments.

As shown, the apparatus 200 includes an electrochemical cell that can electrochemically reduce at least disulfide bonds, sulfur-selenium bonds, diselenide bonds or the like in biochemical compositions such as proteins (e.g., antibodies, enzymes, receptors, etc.). The apparatus 200 can include a porous membrane 1 between a smaller chamber 3 and a larger chamber 5, wherein the smaller chamber 3 corresponds to the half-cell in which a biochemical composition is reduced, and wherein the larger chamber 5 corresponds to the other half-cell of the electrochemical cell. Electrochemical cells use a free flow of electrons from a negative electrode such as a working electrode 2 and a positive electrode such as a counter electrode 6. The porous membrane 1 allows the free flow of electrons to occur while keeping the biochemical composition being reduced inside the smaller chamber 3. Containment in the smaller chamber 3 keeps the biochemical composition close to the working electrode 2 for reduction, as well as keeps the biochemical composition from diluting in the larger chamber 5. The working electrode 2 (e.g., Pt) can be attached to a positive lead of the direct current (“DC”) power supply. It is at a charged surface of the working electrode 2 where, for example, disulfide bonds are reduced in the biochemical composition. It is important that the electrolyte (e.g., buffer solution) including the biochemical composition is agitated or mixed thoroughly throughout an electrochemical reduction, otherwise electrochemical reduction to the desired degree might not be achieved. A magnetic stir bar 4 can be included in the smaller chamber 3 for agitation of the electrolyte including the biochemical composition. It is also important that the volume of the larger chamber 5 be larger than the volume of the smaller chamber 3. For example, the larger chamber 5 can be up to 50× larger (or more) than the smaller chamber 3. In the example provided herein, for example, the larger chamber 5 can be 50 mL, and the smaller chamber 3 can be 3 mL. For better results, the counter electrode 6 (e.g., carbon) should be deeply submerged into the electrolyte (e.g., buffer solution) of the larger chamber 5, and the counter electrode 6 should be larger than the working electrode 2.

In operation, a voltage is applied across the working and counter electrodes of the electrochemical cell to effect, for example, disulfide reduction and yield sulfhydryl groups in the biochemical composition. The apparatus 200 or the electrochemical cell thereof can use a cellulose membrane as the porous membrane 1 to separate the smaller chamber 3 from the larger chamber 5 of the electrochemical cell. The porous membrane 1 (e.g., cellulose membrane) can have pores small enough to keep the biochemical composition in the small chamber 3 while allowing the exchange of electrons between the working electrode 2 in the smaller chamber 3 and the counter electrode 6 in the larger chamber 5. Such pores of the porous membrane 1 can have a molecular weight cutoff from about 3×, 4×, 5×, or 6× smaller than the biomolecules of the biochemical composition. In some embodiments, the porous membrane 1 is a regenerated cellulose membrane with pores sized to retain a biochemical composition including at least a 150 kDa protein. For example, the regenerated cellulose membrane can have a molecular weight cutoff of about 50 kDa.

Chemical reduction methods for biochemical compositions require chemical reducing agents and often extended reaction times. The apparatuses and methods provided herein remove the need for chemical reducing agents and significantly shortens the time needed to reduce biochemical compositions including disulfide bonds, sulfur-selenium bonds, diselenide bonds, which is a crucial first step in creating bioconjugates such as ADCs. The apparatuses and methods provided herein ultimately provide more pure bioconjugates as well. On a laboratory scale, the apparatuses and methods provided herein provide a quick and easy access to multiple bioconjugates without the need to purify away chemical reducing agents. On a large scale, the apparatuses and methods provided herein remove the scaled cost of chemical reduction and speed production of the bioconjugates.

The methods and apparatus disclosed herein relate to electrochemical reduction of disulfide bonds for the purpose of bioconjugation and an electrochemical cell for carrying out such a reduction. Bioconjugation methods are defined as processes for chemically joining two molecules, one of which is a biomolecule and the other a chemical moiety. Bioconjugation is separated into groups based on the active binding site of the chemical moiety which, in some embodiments of the present disclosure, is the sulfhydryl (—SH). The sulfhydryl is typically involved in a disulfide bond (—S—S—) and needs to be made available by reduction of the bond between the two sulfur atoms and this embodiment is a new method to do so.

Advantages of the apparatuses and methods provided herein over chemical reducing agents and methods thereof include, but are not limited to, increased reaction rates, no harsh chemicals, reusability, tunability (and specificity), and integration with certain standards. With respect to increased reaction rates, for example, an electrochemical reduction of disulfide bonds can take minutes versus hours for chemical reduction. With respect to no harsh chemicals, for example, an electrochemical reduction eliminates the need for chemical reducing agents. With respect to reusability, for example, since electrochemical reduction happens at a surface of an electrode, the electrode surface can simply be cleaned and reused. With respect to tunability (and specificity), for example, since electrochemical reduction is based in part on a voltage, the voltage can be customized for each type of protein, as well as certain disulfide bonds, sulfur-selenium bonds, or diselenide bonds within each type of protein. With respect to integration with certain standards, for example, electrochemical reduction can utilize standard formulation buffers for electrolytes. Furthermore, methods of chemical reduction require proteins to be subjected to buffer conditions during conjugation that are different than buffer conditions in the final formulation. This can lead to instability and precipitation. The apparatuses and methods provided herein allows proteins such as antibodies to be in a stable buffer formulation for the duration of the electrochemical process and through bioconjugation.

Bioconjugation takes on many forms and typically involves using a chemically active portion of a biomolecule (either endogenous or artificially created) and creating a chemical linker that can react with this area and tether to the biomolecule a chemical moiety. One such chemically active moiety is the sulfhydryl group (—SH) and, in biomolecules, the only amino acid with this group is cysteine. The cysteine sulfhydryl in most biomolecules is occupied in a disulfide bond (—S—S—) with another cysteine sulfhydryl and the bond between the two groups must be reduced for the chemical linkage of bioconjugation to occur. Historically this has been done with reducing agents such as DTT or TCEP but this method removes these chemical agents from the bioconjugation process altogether and provides additional advantages over this standard bioconjugation process. The standard process requires anywhere from an hour to 24 hours for reduction before conjugation and the final mixture purified to remove the chemical reducing agents. The following are some advantages of the disclosed methods and apparatus:

1. Speed of reduction: minutes versus hours to reduce the disulfide bonds for conjugation
2. No harsh chemicals: The use of electrochemistry eliminates the need for chemical reducing agents
3. Reusability of reduction reagents: Since the reduction happens at the electrode surface it can be rinsed and reused for each reaction.
4. Tunability: Because the reduction is based on a voltage, a value can be set and can be customized per protein.
5. Works with standard formulation buffers: Current reduction strategies require that the protein be subjected to buffer conditions during conjugation that are different than the final formulation leaving the potential to instability and precipitation. Our method allows the protein to be in its stable buffer formulation for the duration of the conjugation process.

In one example, the disclosed method may be implemented for use in the bioconjugation of antibodies for clinical and non-clinical uses, but it can also be used in any conjugation where disulfide reduction is required. This method is especially useful in the clinical arena because it eliminates the need for a reduction chemical in the process of making a drug. This lowers cost and improves safety, which are both invaluable in developing pharmaceutical drugs.

The disclosed method uses an electrochemical cell to apply a voltage to an electrode for disulfide reduction. A further modification of the electrochemical cell uses a cellulose membrane to separate the chambers of the electrochemical cell. This membrane is porous but whose pores are small enough to keep the protein contained in a small volume container while allowing the exchange of electrons between the metal electrode in the small volume and the carbon electrode in the larger volume. Current standard methods only require a container in which to mix the protein, chemical moiety and reducing agent.

On the small scale this method can use more set-up than current methods as you need the electrochemical cell to perform the reduction but on a larger clinical scale the physicality of the two methods is very similar.

This method is adaptable in terms of scale of application and throughput. This method utilizes the flow of electrons which is a scalable phenomenon and it is also very fast which lends itself to higher throughput applications. “Scale” includes volume of the whole reaction.

Current methods require chemicals and time to make the connection between the chemical and the protein. This embodiment removes the need for chemical reducing agents, significantly shortens the time needed to reduce the protein disulfides which is the crucial first step in creating the protein chemical link, and ultimately creates a cleaner final bioconjugate. On a small scale this provides a quick and easy process to create multiple bioconjugates without the need to purify away reducing agents. On a large scale this embodiment removes the scaled cost of disulfide reduction and speeds production of the final bioconjugate.

The FIG. 2A and FIG. 2B depict an example of an electrochemical cell that can perform the invented method described in this document but other similar apparatus could be made that are capable as well but would benefit from a few key components which are labeled in the diagram. Label 1 in FIG. 2A refers to the porous membrane between the smaller chamber marked 3 and the larger chamber marked 5. Electrochemical cells use a free flow of electrons from the negative “working” electrode (marked 2) and the positive “counter” electrode (marked 6) and this membrane allows for this process to occur while keeping the protein being reduced inside the smaller container 3. This containment keeps the protein from becoming diluted and close to the electrode marked 2 for the reduction process. The platinum electrode marked 2 in FIG. 2A is attached to the positive lead of the DC power supply and it is at its charged surface where the disulfide bonds are reduced but it is crucially important that the solution is mixed thoroughly throughout the process by the magnetic stir bar marked 4 otherwise very little to no reduction occurs. It is important to the electrochemical cell that the volume of the container marked 5 be larger than the container marked 3 and in this case it is approximately 17× larger (50 ml vs 3 ml) and that the carbon electrode marked 6 be deeply submerged into the buffer of container 5 and larger than the platinum electrode marked 2.

The process begins by filling the chamber marked 5 in FIG. 2A with the same buffer the protein that is to be reduced is in and the second chamber (marked 3 in the same figure) is suspended above the larger chamber and the cap with a hole in the middle is screwed on. The sample buffer may or may not require a proton donating chemical like glucose or sucrose. A magnetic stir bar (marked 4 in FIG. 2A) is added along with the protein in buffer to the chamber marked 3 in FIG. 2A. The platinum electrode (marked 2 in FIG. 2A) is inserted into the chamber marked 3 so that it is just above the bottom of the chamber. The electrode marked 2 is then attached to the positive or red end of a DC power supply and the carbon electrode marked 6 is attached to the black or negative end of the same DC power supply. The DC power supply is set to 1.5 volts for 8 minutes while a magnetic stir plate positioned next to the whole apparatus spins the magnetic bar stirring the solution (marked 4) in the chamber marked 3. After 2-8 minutes the DC power supply is turned off as well as the magnetic stir plate. The protein solution is removed from the chamber marked 3 and transferred to a new container where the chemical conjugate is added and the conjugation reaction occurs.

Chamber Design:

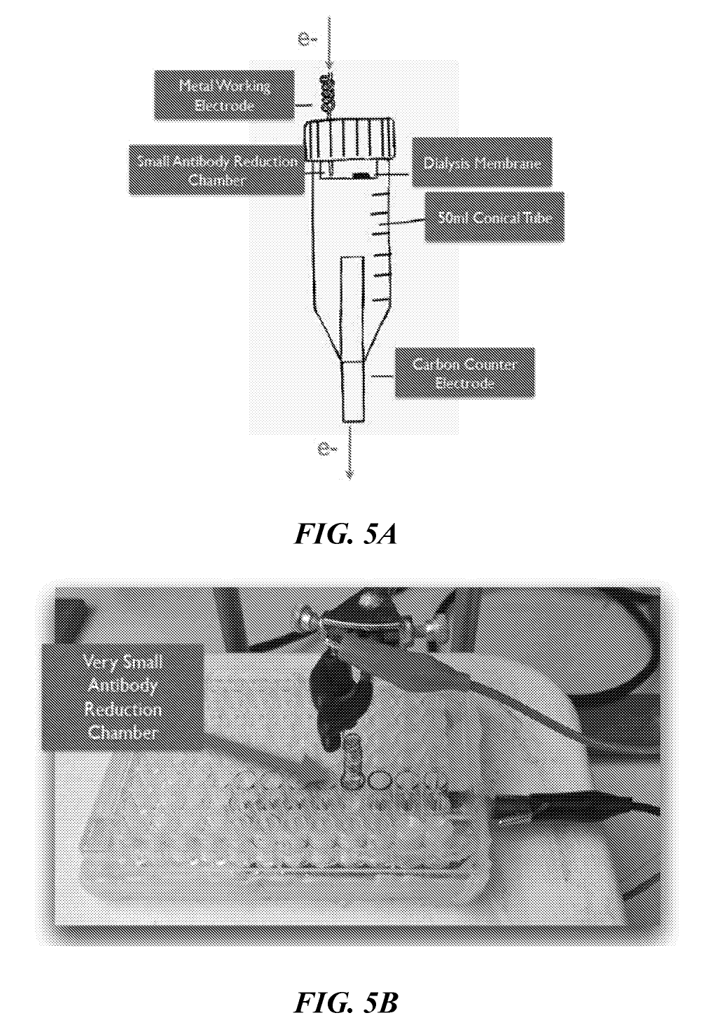

As described above, the electrochemical apparatus of the present disclosure may be configured in a variety of formats and geometries. In some embodiments, for example, the first and second chambers of the device may comprise a cylindrical geometry, and may be aligned in either a concentric or non-concentric manner. FIG. 5A, for example, illustrates one non-limiting example of an electrochemical device comprising a 3 mL reaction chamber and a 50 mL exterior chamber fabricated from a conical centrifugation tube where the two chambers are cylindrical in cross-section and aligned in a concentric manner.

Other chamber geometries are also possible. For example, in some embodiments, the three-dimensional geometry of the first or second chamber may be spherical, cylindrical, elliptical, conical, hemispherical, cubic, rectangular, or polyhedral, or may have an irregular three-dimensional geometry. In some embodiments, the three-dimensional geometry of the first and second chambers may be the same. In some embodiments, the three-dimensional geometries of the first and second chambers may be different.

In some embodiments, the volume of the first or second chamber may range from about 0.01 mL to about 1000 mL. In some embodiments, the volume of the first or second chamber may be at least 0.01 mL, at least 0.1 mL, at least 1 mL, at least 10 mL, at least 100 mL, 1000 mL or greater. In some embodiments, the volume of the first or second chamber may be at most 200 mL, at most 100 mL, at most 10 mL, at most 1 mL, at most 0.1 mL, at most 0.01 mL, or smaller. Any of the lower and upper values described in this paragraph may be combined to form a range included within the disclosure, for example, the volume of the first or second chamber may range from about 0.1 mL to about 10 mL. Those of skill in the art will recognize that first or second chamber may have any value within this range, e.g., about 2.7 mL.

In general, the volume of the second chamber will be larger than that of the first chamber. In some embodiments, the volume of the second chamber may be at least 2.5×, at least 5×, at least 10×, at least 20×, at least 35×, at least 50×, at least 100×, at least 250×, at least 500×, or at least 1000× larger than the volume of the first chamber. Those of skill in the art will recognize that the ratio of second-to-first chamber volumes may have any value within this range, e.g., the volume of the second chamber may be about 12× larger than the volume of the first chamber.

The chambers of the disclosed electrochemical apparatus may be fabricated using any of a variety of fabrication techniques known to those of skill in the art. Examples of suitable fabrication techniques include conventional machining, CNC machining, injection molding, 3D printing, alignment and lamination of one or more layers of laser or die-cut polymer films, or any of a number of microfabrication techniques such as photolithography and wet chemical etching, dry etching, deep reactive ion etching, or laser micromachining.

The chambers may be fabricated using a variety of materials known to those of skill in the art. In general, the one or more materials used to fabricate the chambers should be non-conductive, and should also be resistant to non-specific adsorption of proteins or other biomolecules. Additionally, the choice of material(s) used will often depend on the choice of fabrication technique used, and vice versa. Examples of suitable materials include, but are not limited to, silicon, fused-silica, glass, any of a variety of polymers, e.g. polydimethylsiloxane (PDMS; elastomer), polymethylmethacrylate (PMMA), polycarbonate (PC), polystyrene (PS), polypropylene (PP), polyethylene (PE), high density polyethylene (HDPE), polyimide, cyclic olefin polymers (COP), cyclic olefin copolymers (COC), polyethylene terephthalate (PET), optical adhesive (NOA), epoxy resins, a non-stick material such as teflon (PTFE), or a combination of these materials. In some embodiments, different components of the chambers may be fabricated from different materials

In some embodiments, the electrochemical apparatus of the present disclosure may comprise multiplexed formats, i.e., they may be configured to perform electrochemical reduction of more than one biochemical composition sample in parallel. FIG. 5B illustrates an electrochemical device comprising a plurality of 300 μL reaction chambers fashioned from the wells of a 96-well plate, where a working electrode is inserted into one or more of the wells. The bottom of the wells have been removed and a dialysis membrane is affixed to the bottom of each well. The counter electrode is located in a rectangular bottom chamber fixed to the underside of the 96-well plate. In some embodiments, a single working electrode is inserted into a single well. In some embodiments, two or more working electrodes, e.g., up to 96 working electrodes, are inserted into a corresponding number of wells to perform reduction of disulfide or related bonds in a plurality of biochemical composition samples. In these embodiments, the plurality of biochemical composition samples are preferably suspended in the same electrochemical reduction buffer, and are processed using the same set of voltage and run-time process parameters. In some embodiments, a similar electrochemical apparatus may be configured in a 384-well plate or 1536-well plate format.

Electrode Design:

The first and second electrodes of the disclosed electrochemical apparatus may be configured in a variety of geometries and dimensions, although in general, the second electrode will preferably be larger than the first electrode to promote efficient transfer of electrons through the porous membrane and to ensure that the half-reaction occurring at the second electrode (i.e., the counter electrode) occurs fast enough so as not to limit the reduction process occurring at the working electrode (thereby promoting efficient electrochemical reduction of biochemical compositions placed in the first chamber).

For example, in some embodiments, the electrodes may comprise a cylindrical (e.g., a wire-like or rod-like) geometry. In some embodiments, the electrodes may comprise a rectangular cross-sectional geometry and comprise a bar-like or plate-like three-dimensional geometry. In some embodiments, the electrodes may comprise a mesh or brush-like geometry. In some embodiments, the electrodes may comprise a rotating disk geometry. In some embodiments, the first electrode and the second electrode may have the same geometries. In some embodiments, the first electrode and the second electrode may have different geometries.

The electrodes of the present disclosure may be fabricated from any of a variety of materials and using any of a variety of fabrication techniques known to those of skill in the art.

For example, the first electrode of the disclosed electrochemical apparatus (i.e., the working electrode, which is a negative electrode or cathode) may generally have a wire-like, rod-like, or plate-like three-dimensional geometry and may typically be fabricated from an electrochemically inert metal such as, but not limited to, platinum, gold, silver, titanium, titanium oxide, and the like, or any combination thereof. In some embodiments, the first electrode preferentially does not comprise titanium or titanium oxide.

The second electrode of the disclosed electrochemical apparatus (i.e., the counter electrode, which is a positive electrode or anode) may generally have a wire-like, rod-like, plate-like, mesh-like, or rotating disk geometry, and may be fabricated from carbon, gold, platinum, or a similar electrochemically inert material known to those of skill in the art. In some embodiments, the second electrode is preferentially fabricated from carbon, and may comprise a carbon mesh in a planar, cylindrical, or spiral wound geometry. In some embodiments, the second electrode may be a reticulated vitreous carbon electrode (i.e., a porous carbon electrode).

In some embodiments, the working electrode is carbon, and the counter electrode is platinum.

The dimensions of the first and second electrodes will vary according to the configuration of the electrochemical apparatus and the geometry and dimensions of the first and second chambers. For example, for first or second electrodes having a cylindrical, bar-like, or plate-like geometry, the largest cross-sectional dimension (e.g., the diameter for a cylindrical electrode, or the length or width for a bar-like or plate-like electrode) may range from about 0.1 mm to about 20 mm. In some embodiments, the largest cross-sectional dimension of the first or second electrode may be at least 0.1 mm, at least 0.5 mm, at least 1 mm, at least 2 m, at least 3 mm, at least 4 mm, at least 5 mm, at least 6 mm, at least 7 mm, at least 8 mm, at least 9 mm, at least 10 mm, at least 15 mm, at least 20 mm, or larger. In some embodiments the largest cross-sectional dimension of the first or second electrode may be at most 20 mm, at most 15 mm, at most 10 mm, at most 9 mm, at most 8 mm, at most 7 mm, at most 6 mm, at most 5 mm, at most 4 mm, at most 3 mm, at most 2 mm, at most 1 mm, at most 0.5 mm, at most 0.1 mm, or smaller. Any of the lower and upper values described in this paragraph may be combined to form a range included within the disclosure, for example, the largest cross-sectional dimension of the first or second electrode may range from about 1 mm to about 9 mm. Those of skill in the art will recognize that the largest cross-sectional dimension for the first or second electrode may have any value within this range, e.g., about 7.5 mm.

In some embodiments, the length (or longest non-cross-section dimension) for the first or second electrode may range from about 1 mm to about 100 mm. In some embodiments, the length of the first or second electrode may be at least 1 mm, at least 5 mm, at least 10 mm, at least 20 mm, at least 30 mm, at least 40 mm, at least 50 mm, at least 60 mm, at least 70 mm, at least 80 mm, at least 90 mm, at least 100 mm, or larger. In some embodiments the length of the first or second electrode may be at most 100 mm, at most 90 mm, at most 80 mm, at most 70 mm, at most 60 mm, at most 50 mm, at most 40 mm, at most 30 mm, at most 20 mm, at most 10 mm, at most 5 mm, at most 1 mm, or smaller. Any of the lower and upper values described in this paragraph may be combined to form a range included within the disclosure, for example, the length of the first or second electrode may range from about 5 mm to about 80 mm. Those of skill in the art will recognize that the length of the first or second electrode may have any value within this range, e.g., about 88 mm.

Porous Membranes:

The porous membrane used to separate the first and second chambers of the disclosed electrochemical apparatus may comprise any of a variety of membrane or membrane-like materials known to those of skill in the art. Examples include, but are not limited to, dialysis membranes (e.g. cellulose or cellulose ester membranes, polyethylene membranes, polypropylene membranes, polysulfone membranes, polyethersulfone (PES) membranes, polytetrafluoroethylene (PTFE) membranes, etched polycarbonate membranes, or collagen membranes), porous glass (e.g., controlled pore glass), and the like. In some preferred embodiments, the porous membrane is a cellulose membrane.

The porous membrane allows electrons and ions to pass while retaining biomolecules that are larger than the average pore size. In some embodiments, the average pore diameter of the porous membrane may range from about 10 angstroms to about 200 angstroms. In some embodiments, the average pore diameter may be at least 10 angstroms, at least 20 angstroms, at least 30 angstroms, at least 40 angstroms, at least 50 angstroms, at least 60 angstroms, at least 70 angstroms, at least 80 angstroms, at least 90 angstroms, at least 100 angstroms, at least 120 angstroms, at least 140 angstroms, at least 160 angstroms, at least 180 angstroms, at least 200 angstroms, or larger. In some embodiments, the average pore diameter of the porous membrane may be at most 200 angstroms, at most 180 angstroms, at most 160 angstroms, at most 140 angstroms, at most 120 angstroms, at most 100 angstroms, at most 90 angstroms, at most 80 angstroms, at most 70 angstroms, at most 60 angstroms, at most 50 angstroms, at most 40 angstroms, at most 30 angstroms, at most 20 angstroms, at most 10 angstroms, or smaller. Any of the lower and upper values described in this paragraph may be combined to form a range included within the disclosure, for example, the average pore diameter may range from about 30 angstroms to about 120 angstroms. Those of skill in the art will recognize that the average pore diameter may have any value within this range, e.g., about 95 angstroms.

In some embodiments, the molecular weight cut-off for the porous membrane may range from about 10 kDa to about 200 kDa. In some embodiments, the molecular weight cut-off may be at least 10 kDa, at least 20 kDa, at least 30 kDa, at least 40 kDa, at least 50 kDa, at least 60 kDa, at least 70 kDa, at least 80 kDa, at least 90 kDa, at least 100 kDa, at least 125 kDa, at least 150 kDa, at least 175 kDa, at least 200 kDa, or higher. Those of skill in the art will recognize that the molecular weight cut-off of the porous membrane may have any value within this range, e.g., about 66 kDa.

Power Supplies:

The first and second electrodes of the disclosed electrochemical apparatus are configured to work together to electrochemically reduce one or more biochemical compositions placed in the first chamber when voltage is applied across the first and second electrodes. Any of a variety of DC power supplies known to those of skill in the art may be used. Examples of suitable DC power supplies include, but are not limited to, the Keithley Series 2220/2230/2231 DC power supplies, or the Keithley Series 2300 DC power supplies (Tektronix, Beaverton, Oreg.). In some embodiments, the first electrode may be the negative electrode and the second electrode may be the positive electrode. In some embodiments, the first electrode may be the positive electrode and the second electrode may be the negative electrode. In some embodiments, the first and second electrodes of the disclosed electrochemical apparatus are configured to work together to electrochemically oxidize one or more biochemical compositions placed in the first chamber when voltage is applied across the first and second electrodes.

Voltage Settings, Current Settings, & Process Times:

Adjustment of the voltage settings and process times used with the disclosed methods and electrochemical apparatus allow one to optimize the reduction process and control the extent of the reaction. Examples of suitable voltage settings may range from about 0.1 V to about 10 V. In some embodiments, the voltage setting may be at least 0.1 V, at least 0.5 V, at least 1 V, at least 1.5 V, at least 2 V, at least 2.5 V, at least 3 V, at least 4 V, at least 5 V, at least 10 V, or higher. In some embodiments, the voltage setting may be at most 10 V, at most 5 V, at most 4 V, at most 3V, at most 2.5 V, at most 2 V, at most 1.5 V, at most 1 V, at most 0.5 V, or at most 0.1 V. Any of the lower and upper values described in this paragraph may be combined to form a range included within the disclosure, for example, the voltage setting may range from about 1.5 V to about 4 V. Those of skill in the art will recognize that the voltage setting may have any value within this range, e.g., about 2.6 V. The optimal voltage setting may vary depending on a variety of experimental and apparatus design parameters, e.g., the dimensions of the first and second chamber, the dimensions and geometries of the first and second electrodes, the pore size and pore packing density (and hence the electrical resistance) of the porous membrane, the conductivity of the buffer, etc. Use of excessively high voltage settings may result in large pH changes and/or biomolecule degradation.

In some cases, current settings may be used to control the electrochemical process rather than voltage settings. Examples of suitable current settings may range from about 0.1 mA to about 1 A. In some embodiments, the current setting may be at least 0.1 mA, at least 0.5 mA, at least 1 mA, at least 1.5 mA, at least 2 mA, at least 2.5 mA, at least 3 mA, at least 4 mA, at least 5 mA, at least 10 mA, at least 100 mA, at least 200 mA, at least 300 mA, at least 400 mA, at least 500 mA, at least 600 mA, at least 700 mA, at least 800 mA, at least 900 mA, at least 1 A, or higher. In some embodiments, the current setting may be at most 1 A, at most 900 mA, at most 800 mA, at most 700 mA, at most 600 mA, at most 500 mA, at most 400 mA, at most 300 mA, at most 200 mA, at most 100 mA, at most 10 mA, at most 5 mA, at most 4 mA, at most 3 mA, at most 2.5 mA, at most 2 mA, at most 1.5 mA, at most 1 mA, at most 0.5 mA, or at most 0.1 mA. Any of the lower and upper values described in this paragraph may be combined to form a range included within the disclosure, for example, the current setting may range from about 1.5 mA to about 10 mA. Those of skill in the art will recognize that the current setting may have any value within this range, e.g., about 2.6 mA. The optimal current setting may vary depending on a variety of experimental and apparatus design parameters, e.g., the dimensions of the first and second chamber, the dimensions and geometries of the first and second electrodes, the pore size and pore packing density (and hence the electrical resistance) of the porous membrane, the conductivity of the buffer, etc. Use of excessively high current settings may result in large pH changes and/or biomolecule degradation.

Examples of suitable process times may range from about 0.1 minute to about 100 minutes. In some embodiments, the process time may be at least 0.1 minute, at least 0.25 minutes, at least 0.5 minutes, at least 0.75 minutes, at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 20 minutes, at least 30 minutes, at least 40 minutes, at least 50 minutes, at least 60 minutes, at least 70 minutes, at least 80 minutes, at least 90 minutes, at least 100 minutes, or longer. In some embodiments, the process time may be at most 100 minutes, at most 90 minutes, at most 80 minutes, at most 70 minutes, at most 60 minutes, at most 50 minutes, at most 40 minutes, at most 30 minutes, at most 20 minutes, at most 10 minutes, at most 9 minutes, at most 8 minutes, at most 7 minutes, at most 6 minutes, at most 5 minutes, at most 4 minutes, at most 3 minutes, at most 2 minutes, at most 1 minutes, at most 0.5 minutes, at most 0.1 minutes, or shorter. Any of the lower and upper values described in this paragraph may be combined to form a range included within the disclosure, for example, the process time may range from about 2 minutes to about 4 minutes. Those of skill in the art will recognize that the process time used may have any value within this range, e.g., about 3.5 minutes.

Electrochemical Reduction Buffers:

Any of a variety of buffers known to those of skill in the art may be used to perform the disclosed methods. One non-limiting example is phosphate buffered saline (PBS) buffered at pH 7.4, and including 10 mM EDTA and 10 mM glucose. Glucose is an important buffer component as it serves as a proton donor for the reduction reaction. Glucose (or an alternative proton donor such as sucrose) may be included at a concentration of at least 0.1 mM, 0.5 mM, at least 1 mM, at least 1.5 mM, at least 2 mM, at least 2.5 mM, at least 5 mM, at least 7.5 mM, at least 10 mM, at least 15 mM, or at least 20 mM. Those of skill in the art know that the glucose concentration (or the concentration of an alternative proton donor) used for electrochemical reduction may have any concentration in this range, e.g. about 0.8 mM. EDTA prevents re-formation of disulfide bonds following reduction, and may be used at a concentration similar to that used for glucose.

Agitation Methods:

An important feature of the disclosed electrochemical reduction apparatus is the inclusion of a magnetic stir bar or other agitation mechanism. Vigorous agitation of the solution in the first chamber comprising the biochemical composition to be reduced is important for efficient reduction, as it serves to replenish the supply of the biochemical composition at the surface of the working electrode. Examples of suitable agitation mechanisms (parts of which may be incorporated into the electrochemical apparatus itself, and parts of which may be external to the electrochemical apparatus) include, but are not limited to, magnetic stir bars or magnetic beads and their associated magnetic field controllers, shaft-driven plastic propellers or impellers, and the like.

### EXAMPLES

These examples are provided for illustrative purposes only and not to limit the scope of the claims provided herein.

### Example 1: Electrochemical Device with Three Milliliter Reduction Chamber

The electrochemical device is in a three milliliter reduction chamber volume format. The electrochemical device includes an exterior chamber, an interior chamber, a working electrode, a counter electrode, an electrolyte, and a proton-donating agent. The outer chamber has a larger volume than the interior chamber. The volume of the exterior chamber is 50 milliliters (mL). The volume of the interior chamber is 3 mL. The bottom portion of the interior chamber is a porous membrane. The sample to be reduced is contained within the interior chamber. Both the internal and external chambers contain an identical electrolyte and proton-donating agent. The working electrode is platinum and the counter electrode is carbon. During operation of the electrochemical device, an electrical potential is applied across the working and counter electrodes. The porous membrane of the interior chamber allows for the movement of electrons across the membrane while maintaining the sample within the interior chamber. FIG. 5A shows an illustration of an electrochemical device with a 3 mL reaction chamber. The exterior chamber is a 50 mL conical centrifugation tube. The counter electrode is inserted through a hole at the bottom of the exterior chamber. The interior chamber is inserted into the exterior chamber at the top of the chamber and a lid is applied to cover both chambers. The working electrode is inserted into the interior chamber through a hole in the lid.

### Example 2: Electrochemical Device with Three Hundred Microliter Reduction Chamber

The electrochemical device is in a three hundred microliter (μL) reduction chamber volume format. The electrochemical device includes a top chamber, a bottom chamber, a working electrode, a counter electrode, an electrolyte, and a proton-donating agent. The top chamber is the well of a 96-well plate with a 300 μL reaction volume. The bottom of the well is removed by mechanical drilling and a porous dialysis membrane is affixed to the underside of the 96-well plate. A water tight 150 ml bottom chamber is constructed around the bottom of the 96-well plate. The working electrode is inserted into the top chamber. The counter electrode is inserted into the bottom chamber. FIG. 5B shows an electrochemical device with a 300 μL reaction chamber. The top chamber is a single well of a 96-well plate and the working electrode is inserted into the well. The bottom of the well has been removed by mechanical drilling and a dialysis membrane is affixed to the well bottom. The counter electrode is located in the bottom chamber. The sample to be reduced is added to the well of the chamber. During electrochemical reduction, ions pass through the dialysis membrane and the sample is maintained within the well.

### Example 3: Disulfide Bond Reduction in a Monoclonal Antibody Fragment

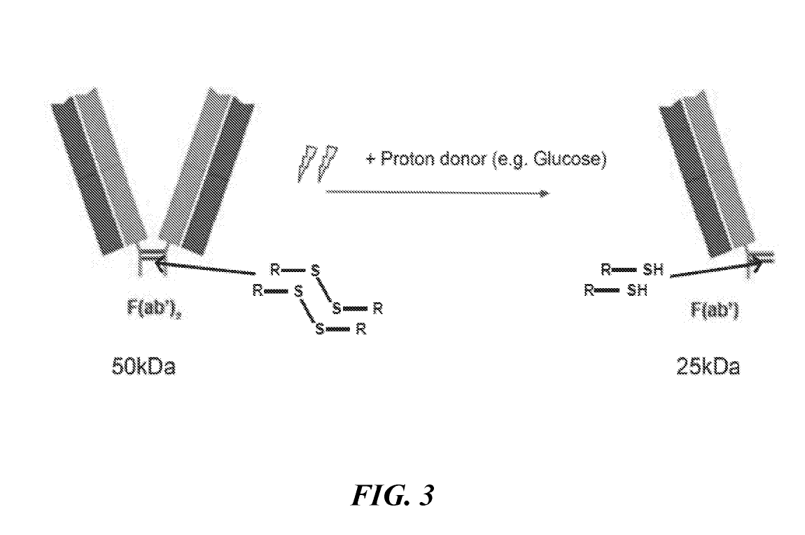

Disulfide bonds present in monoclonal antibody fragments are reduced in the electrochemical device. The monoclonal antibody fragment comprises a bivalent 50 kDa antigen-binding fragment (F(ab′)₂). The F(ab′)₂ has disulfide bonds binding the two antigen-binding domains and binding the light chain fragments and heavy chain fragments. FIG. 3 illustrates an example electrochemical reduction of the disulfide bonds connecting the antigen-binding domains in a F(ab′)₂ fragment of an antibody.

The electrochemical device comprises an exterior chamber and an interior chamber. The exterior chamber is a 50 milliliter (mL) conical centrifuge tube with a carbon electrode inserted through a hole in the bottom of the tube. The tube is sealed around the carbon electrode with hot glue. The interior chamber is a 3 mL dialysis unit (e.g., a ThermoFisher Scientific Slide-A-Lyzer™ MINI Dialysis Device, Waltham, Mass.) with a regenerating cellulose membrane. The electrochemical device is prepared by filling the exterior chamber with phosphate buffered saline (PBS) at pH 7.4 and filling the interior chamber PBS buffer, the F(ab′)₂ fragment to be reduced, a proton-donating agent, and a magnetic stir bar. The proton-donating agent is glucose, sucrose, or another disaccharide. The interior chamber is suspended in the exterior chamber and the lid of the conical centrifuge tube is screwed onto the exterior chamber to secure the chamber. The lid of the exterior chamber contains a hole. A platinum electrode is inserted through the hole into the interior chamber so that the tip of the electrode is positioned just above the cellulose membrane at the bottom of the interior chamber. The platinum electrode is attached to the positive side of a DC power supply and the carbon electrode is attached to the negative side of a DC power supply. A constant voltage is supplied to the electrochemical device while the magnetic stir bar mixes the solution in the interior chamber. In one trial, 3 volts (V) of electrical potential is supplied to the electrochemical cell for thirty minutes. In another trial, 1.5 V of electrical potential is supplied to the electrochemical cell for thirty minutes. In both trials, samples are taken every two minutes to test for reduction of the disulfide bonds. After the allotted time period, the voltage is turned off and the mAb fragment is removed for post-reduction processing.

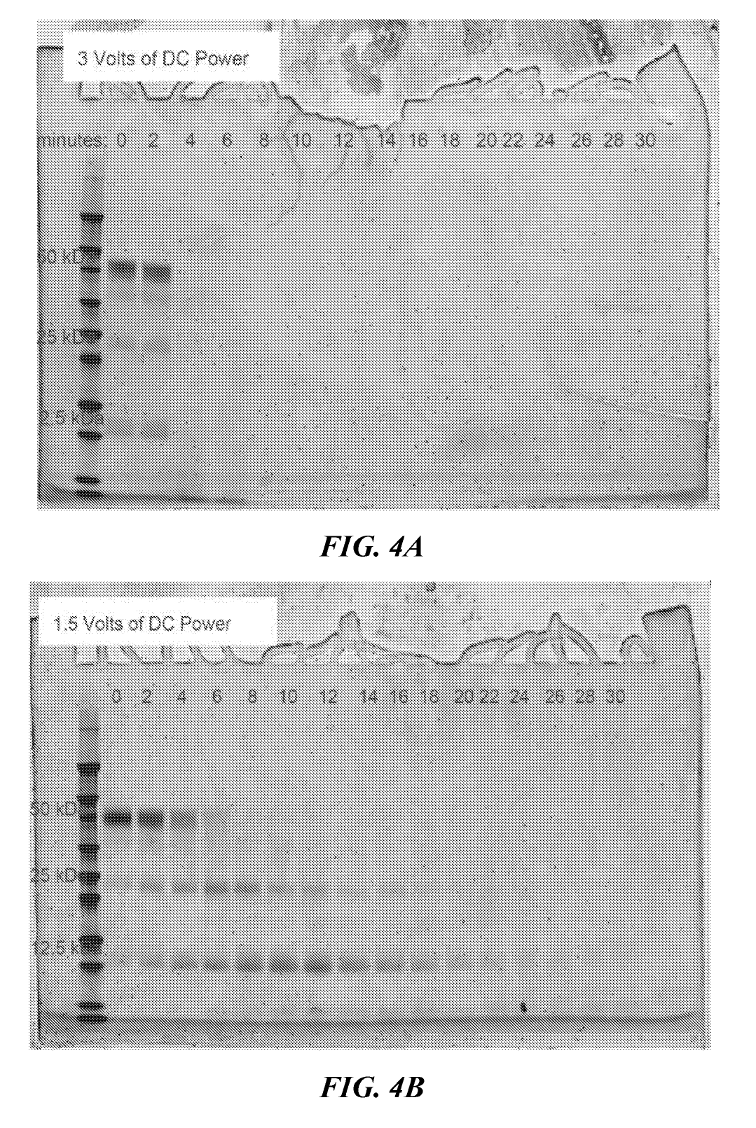

The reduction of the mAb fragment is determined by gel electrophoresis. FIG. 4A shows the electrophoretogram for a F(ab′)₂ electrochemically reduced at 3 V. The left-most lane of the gel is the size reference ladder. Each subsequent lane is a sample taken during the two minute time intervals. Samples taken at zero and two minutes contain a substantial portion of a 50 kDa F(ab′)₂ and a small portion of both a 25 kDa F(ab′) and a 12.5 kDa fragment. Samples taken between four minutes and thirty minutes show complete degradation of the mAb fragments. FIG. 4B shows the electrophoretogram for a F(ab′)₂ electrochemically reduced at 1.5 V. The left-most lane of the gel is the size reference ladder. Each subsequent lane is a sample taken during the two minute time intervals. The samples taken at zero and two minutes contain a substantial portion of a 50 kDa F(ab′)₂ fragment and a small portion of both a 25 kDa F(ab′) and a 12.5 kDa fragment. The samples taken at four minutes and six minutes show a reduced amount of the F(ab′)₂ fragment and increased amounts of the F(ab′) and 12.5 kDa fragments. The sample taken at eight minutes shows a negligible amount of F(ab′)₂ and a further increase in the amount of the F(ab′) and the 12.5 kDa fragments. The F(ab′)₂ is completely degraded after approximately twenty minutes.

### Example 4: Tuning of Applied Voltage for Electrochemical Reduction

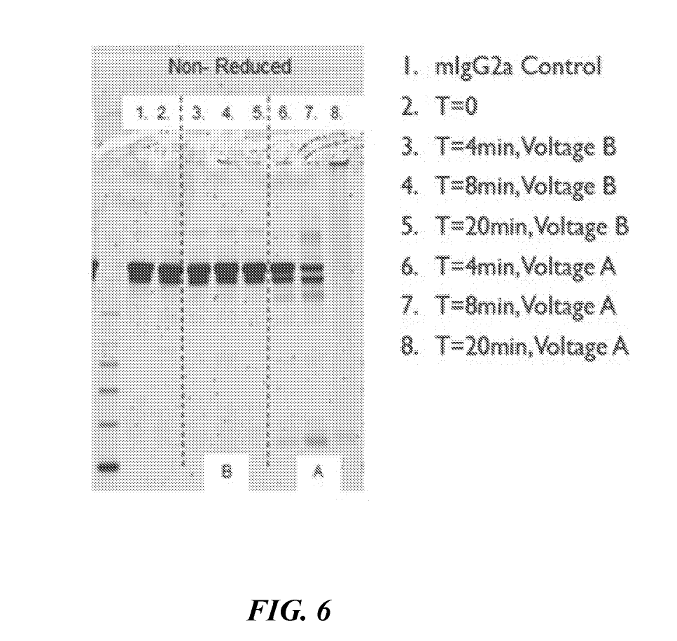

The applied voltage is tuned during electrochemical reduction to enable disulfide reduction without increasing sample aggregation or degradation. The samples to be reduced contain Mouse IgG2a isotype control (mIgG2a) antibody and are reduced using the electrochemical device described in Example 1. The samples are reduced in a PBS, 10 mM EDTA, 10 mM glucose, pH 7.4 buffer where glucose acts as a proton-donating agent. One sample is reduced with 1.5 V of electrical potential applied for twenty minutes. Another sample is reduced with 3 V of electrical potential applied for twenty minutes. Aliquots of the samples are taken at zero, four, eight, and twenty minutes. Sample reduction is determined by gel electrophoresis of 2 micrograms (μg) of sample on a bis(2-hydroxyethyl)amino-tris(hydroxymethyl)methane (Bis-Tris) gel ran with a 2-(N-morpholino)ethanesulfonic acid (MES) buffer. FIG. 6 is an electrophoretogram of a mIgG2a sample reduced at 1.5 V (voltage B) for twenty minutes and a mIgG2a antibody reduced at 3 V (voltage A) for twenty minutes. The left most lane is the size reference ladder. Lane 1 is a non-reduced mIgG2a control. Lanes two, three, four, and eight are mIgG2a samples reduced at 1.5 V for zero, four, eight, and twenty minutes, respectively. Lanes six, seven, and eight are mIgG2a samples reduced at 3 V for four, eight, and twenty minutes, respectively. Samples reduced at 1.5 V are not reduced over the twenty minutes voltage is applied. Samples reduced at 3 V show partial degradation at approximately eight minutes and complete degradation at twenty minutes.

### Example 5: Detection of Free Sulfhydryls after Electrochemical Reduction

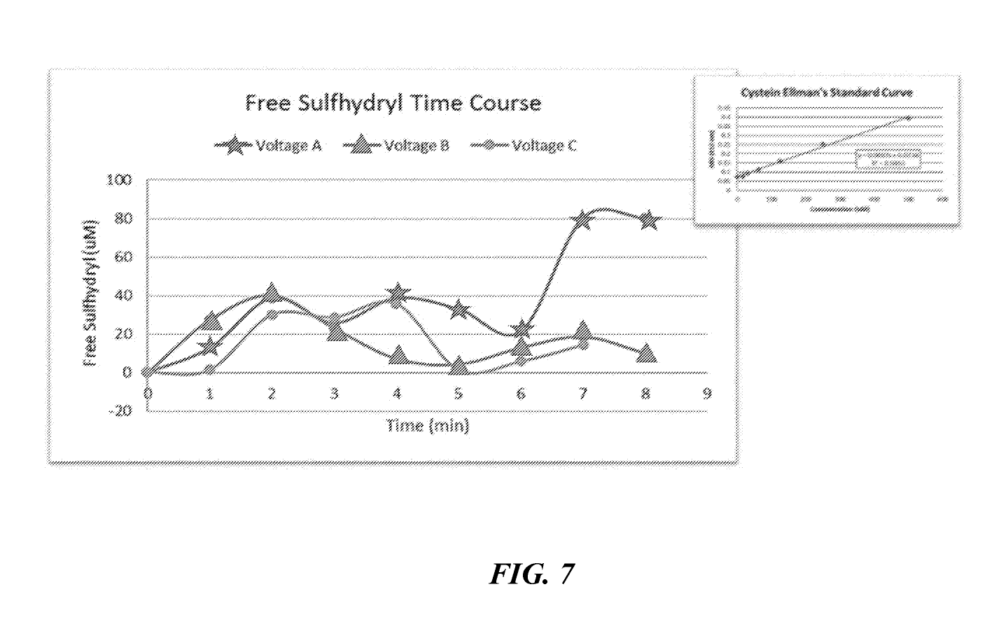

Reduction is verified by the presence of free sulfhydryls after electrochemical reduction is performed. Free sulfhydryls are detected with 5,5-dithio-bis-(2-nitrobenzoidic acid) (DTNB). DTNB reacts with sulfhydryl-containing compounds to produce a yellow color that absorbs at 412 nanometers (nm) and does not react with disulfide-containing compounds. Cystein, an amino acid which contains a free sulfhydryl, is used to generate a standard curve. The standard curve is used to calculate the measured free sulfhydryl concentration in the sample of interest. The samples to be reduced contain Mouse IgG2a isotype control (mIgG2a) antibody and are reduced using the electrochemical device described in Example 1. The samples are reduced in a PBS, 10 mM EDTA, 10 mM glucose, pH 7.4 buffer where glucose acts as a proton-donating agent. One sample is reduced with 1.5 V (voltage B) of electrical potential applied. One sample is reduced with 3 V (voltage A) of electrical potential applied. Another sample is reduced with voltage ranging from 1.5 V to 2.5 V (voltage C) and a constant current of 2 milliamps (mA). Aliquots of the samples are taken every minute for eight minutes. FIG. 7 shows the free sulfhydryl concentration for samples reduced under voltage A, voltage B, and voltage C conditions as a function of time. Samples reduced with voltage A conditions show in increasing concentration of free sulfhydryls as a function of time for zero to eight minutes. Samples reduced with voltage B and voltage C conditions show a changing concentration of free sulfhydryl groups from zero to eight minutes.

### Example 6: Detection of Bioconjugation Efficiency after Electrochemical Reduction

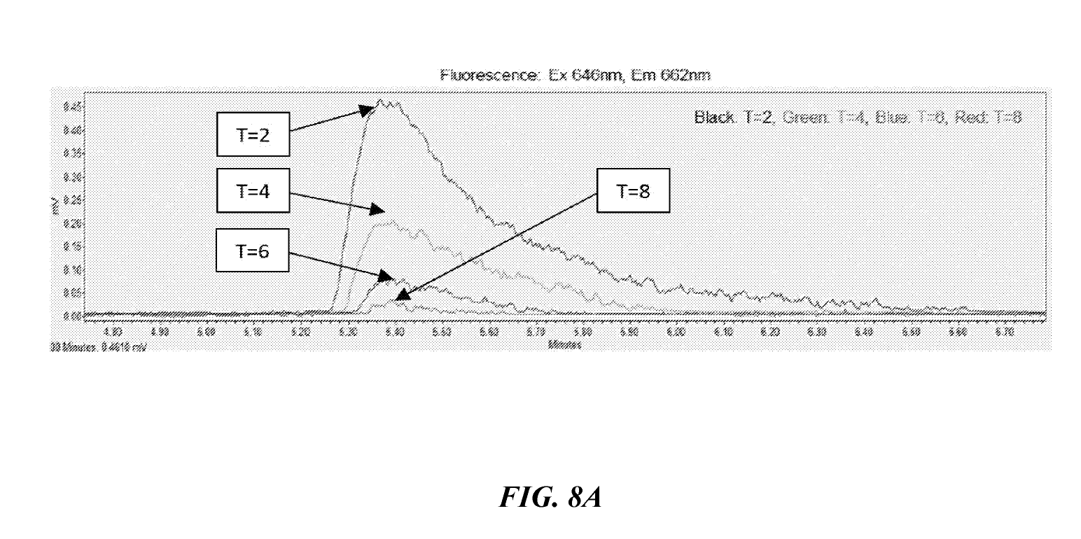

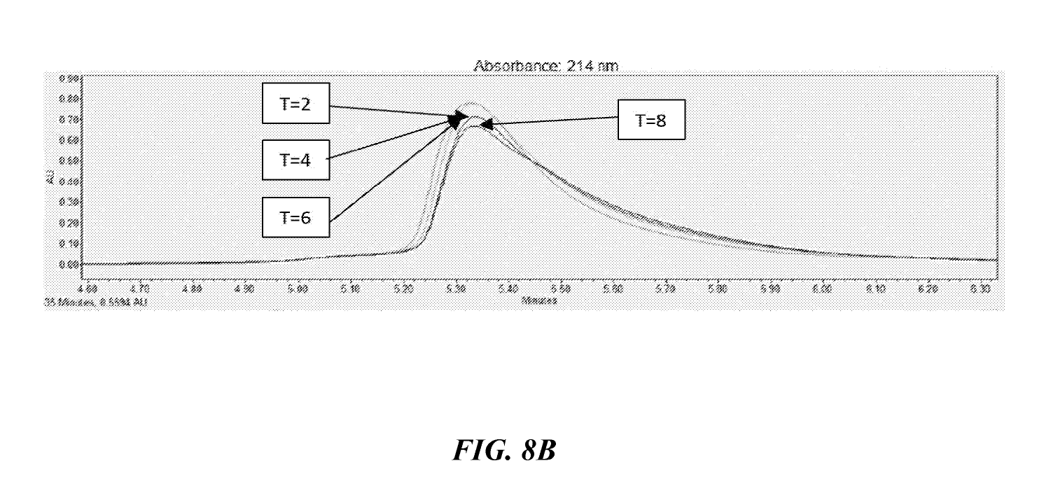

Electrochemical reduction reduces disulfide bonds and frees sulfhydryl groups for bioconjugation reactions. A sample to be reduced contains mIgG2a antibody and is reduced using the electrochemical device described in Example 1. The samples are reduced in a PBS, 10 mM EDTA, 10 mM glucose, pH 7.4 buffer where glucose acts as a proton-donating agent. The sample is reduced with 1.5 V of electrical potential. Aliquots of the sample are taken at two, four, six, and eight minutes. Free sulfhydryls in the mIgG2a antibody are reacted with maleimide-cyanine5, a sulfhydryl reactive linker coupled to a fluorophore. The aliquots are purified using a PD10 de-salting column to remove excess maleimide-cyanine5. High pressure liquid chromatography (HPLC) with a hydrophobic interaction column is used to detect conjugation of the fluorophore to the antibody and total antibody concentration. FIG. 8A shows a chromatograph of fluorophore conjugated mIgG2a, after reduction at 1.5 V of electrical potential, as a function of time. The excitation wavelength is 646 nm and the emission wavelength is 662 nm. The aliquot taken at two minutes shows the largest concentration of fluorophore conjugated to the antibody. The concentration of fluorophore conjugated antibody decreases with each time point until eight minutes. The aliquot taken at eight minutes shows the least amount of fluorophore conjugated to the antibody. FIG. 8B shows a chromatograph of total mIgG2a concentration, after reduction at 1.5 V of electrical potential, as a function of time. The detection wavelength is 214 nm. The chromatograph shows no peak shift and the area under the curve is approximately constant for all time points, indicating that substantial degradation of the antibody has not occurred.

### Example 7: Comparison of Electrochemical and Chemical Reduction

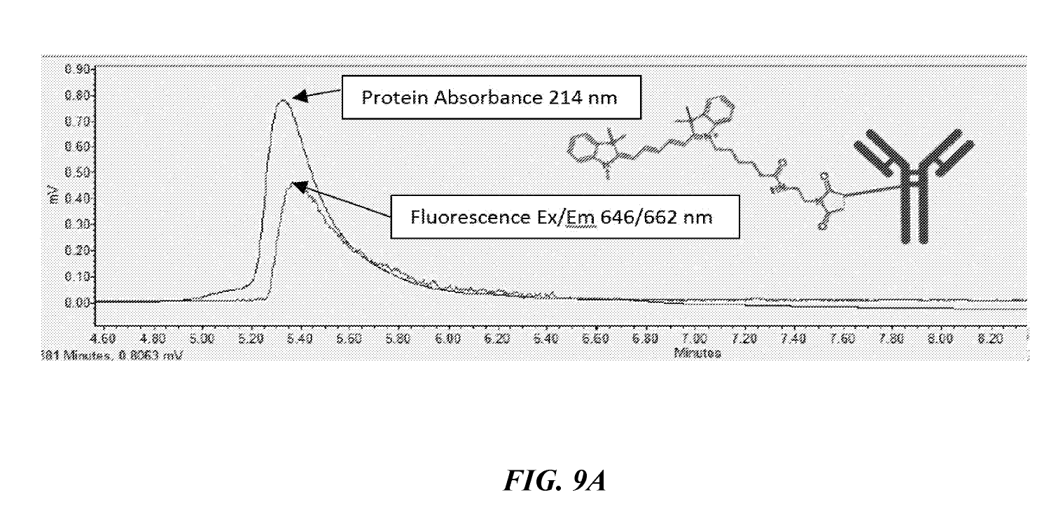

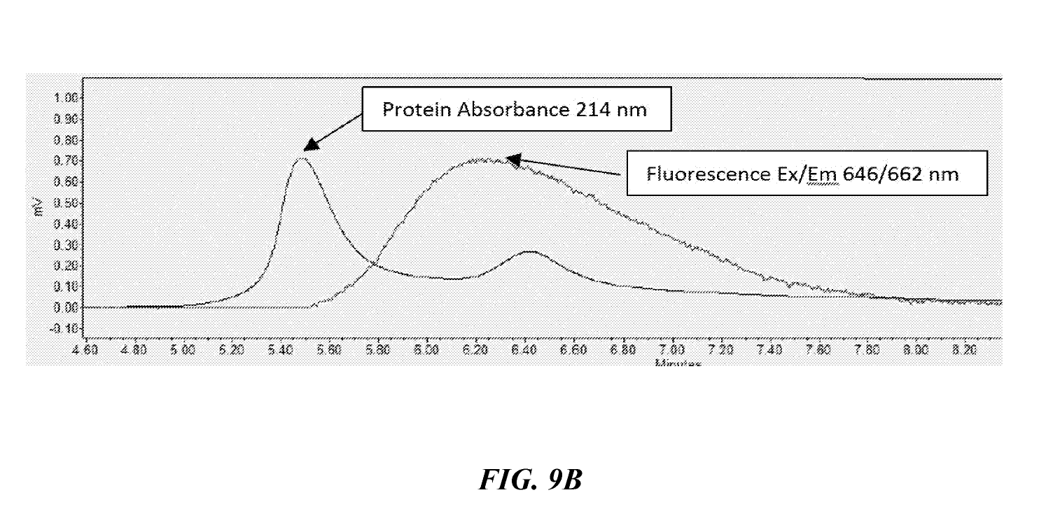

The conjugation efficiency and antibody degradation is compared after electrochemical and chemical reduction techniques. The electrochemical reduction was performed as described in Example 6 by applying 1.5 V of electrochemical potential for two minutes. The free sulfhydryls in the sample are reacted with a maleimide-cyanine5 fluorophore. The sample is purified using a PD10 de-salting column to remove non-conjugated fluorophore. HPLC is used to detect conjugated fluorophore concentration and antibody concentration. Another sample is chemically reduced with a 10:1 molar ratio of tris(2-carboxyethyl)phosphine (TCEP) to mIgG2a for 30 minutes, then the sulfhydryl reactive maleimide-cyanine5 fluorophore at a 5:1 molar ratio and incubated overnight and the sample is purified using a PD10 de-salting column to remove excess fluorophore. FIG. 9A shows an HPLC chromatograph for the electrochemically reduced sample with both a fluorescence trace to detect fluorophore concentration and an absorbance trace to detect antibody concentration. FIG. 9B shows an HPLC chromatograph for the chemically reduced sample with both a fluorescence trace and an absorbance trace. The electrochemically reduced sample shows a single narrow peak for both the conjugated fluorophore concentration and the protein concentration. The chemically reduced sample shows a secondary concentration peak indicating a non-homogenous sample. The chemically reduced sample also shows a broad fluorescence peak.

While the foregoing concepts and embodiments thereof have been provided in considerable detail, it is not the intention of the applicant(s) for the concepts and embodiments provided herein to be limiting. Additional adaptations and/or modifications are possible, and, in broader aspects, these adaptations and/or modifications are also encompassed. Accordingly, departures may be made from the foregoing concepts and embodiments without departing from the scope afforded by the following claims, which scope is only limited by the claims when appropriately construed.

What is claimed is:

1. An electrochemical device comprising:
(a) a first chamber comprising an inserted first electrode;
(b) a second chamber comprising an inserted second electrode; and
(c) a porous membrane which separates the first and second chamber and is configured to provide electrochemical communication between the first chamber and the second chamber,
wherein the first and second electrodes are configured to work together to electrochemically reduce one or more biochemical compositions placed in the first chamber when voltage is applied across the first and second electrodes.

2. The electrochemical device of claim 1, wherein the volume of the second chamber is larger than the volume of the first chamber.

3. The electrochemical device of claim 2, wherein the first chamber is contained within the second chamber.

4. The electrochemical device of any one of claims 1 to 3, wherein the volume of the second chamber is at least 5×, at least 10×, at least 20×, at least 35×, at least 50×, at least 100×, at least 250×, at least 500×, or at least 1000× larger than the volume of the first chamber.

5. The electrochemical device of any one of claims 1 to 4, wherein the volume of the first chamber is about 3 mL and the volume of the second chamber is about 50 mL.

6. The electrochemical device of any one of claims 1 to 4, wherein the volume of the first chamber is about 300 μL, and the volume of the second chamber is at least 150 mL.

7. The electrochemical device of claim 6, wherein the first chamber is comprised of a well in a 96-well plate, and the second chamber is a rectangular chamber attached to the bottom of the 96-well plate, wherein the second chamber is in electrochemical communication with each well in the 96-well plate by means of a porous membrane that separates each well from the rectangular chamber.

8. The electrochemical device of claim 7, wherein the device is configured to provide a first electrode to two or more wells of the 96-well plate, and a single second electrode to the rectangular chamber, such that two or more samples of a biochemical composition may be reduced in parallel.

9. The electrochemical device of claim 8, wherein the two or more samples comprise different biochemical compositions.

10. The electrochemical device of any one of claims 1 to 9, wherein the first chamber contains a magnetic stir bar configured to agitate the one or more biochemical compositions.

11. The electrochemical device of any one of claims 1 to 10, wherein the porous membrane is a cellulose membrane.

12. The electrochemical device of any one of claims 1 to 11, wherein the porous membrane comprises a pore size small enough to retain the biochemical composition.

13. The electrochemical device of claim 12, wherein the biochemical composition is a protein of molecular weight about 150 kDa.

14. The electrochemical device of claim 12, wherein the biochemical composition is a protein of molecular weight about 50 kDa.

15. The electrochemical device of any one of claims 1 to 14, wherein the first electrode is a negative electrode (or cathode).

16. The electrochemical device of any one of claims 1 to 15, wherein the first electrode is a working electrode.

17. The electrochemical device of claim 15 or claim 16, wherein the negative electrode comprises platinum.

18. The electrochemical device of any one of claims 1 to 17, wherein the first electrode does not comprise titanium.

19. The electrochemical device of claim 16, wherein the working electrode is connected to a direct current power supply.

20. The electrochemical device of any one of claims 1 to 19, wherein the second electrode is a positive electrode (or anode).

21. The electrochemical device of any one of claims 1 to 20, wherein the second electrode is a counter electrode.

22. The electrochemical device of claim 16, wherein the positive electrode comprises carbon.

23. The electrochemical device of any one of claims 1 to 22, wherein the biochemical composition is a protein.

24. The electrochemical device of claim 23, wherein the protein is an antibody or an antibody fragment.

25. The electrochemical device of any one of claims 1 to 24, wherein the first chamber and the second chamber comprise a buffer.

26. The electrochemical device of claim 25, wherein the buffer comprises PBS.

27. The electrochemical device of claim 25, wherein the buffer comprises glucose and EDTA.

28. The electrochemical device of claim 25, wherein the first electrode and second electrode are submerged in the buffer.

29. The electrochemical device of any one of claims 1 to 28, wherein the first and second electrodes are configured to work together to electrochemically reduce disulfide, sulfur-selenium bonds, diselenide bonds, or a combination thereof, of the biochemical composition.

30. A method for reducing disulfide bonds, sulfur-selenium bonds, or diselenide bonds in a biochemical composition comprising:
(a) inserting the biochemical composition into an electrochemical cell comprising
(i) a first chamber comprising an inserted first electrode;
(ii) a second chamber comprising an inserted second electrode; and
(iii) a porous membrane which separates the first and second chamber and is configured to provide electrochemical communication between the first chamber and the second chamber,
(b) agitating the biochemical composition with a magnetic stir bar;
(c) applying a voltage across the first and second electrodes of the electrochemical cell, thereby reducing the disulfide bonds, sulfur-selenium bonds, or diselenide bonds of the biochemical composition.

31. The method of claim 30, wherein the voltage is 3 V.

32. The method of claim 30, wherein the voltage is 1.5 V.

33. The method of any one of claims 30 to 32, wherein the volume of the second chamber is larger than the volume of the first chamber.

34. The method of any one of claims 30 to 33, wherein the first chamber is contained within the second chamber.

35. The method of any one of claims 30 to 34, wherein the volume of the second chamber is at least 5×, at least 10×, at least 20×, at least 35×, at least 50×, at least 100×, at least 250×, at least 500×, or at least 1000× larger than the volume of the first chamber.

36. The method of any one of claims 30 to 35, wherein the volume of the first chamber is about 3 mL and the volume of the second chamber is about 50 mL.

37. The method of any one of claims 30 to 35, wherein the volume of the first chamber is about 300 μL, and the volume of the second chamber is at least 150 mL.

38. The method of claim 37, wherein the first chamber is comprised of a well in a 96-well plate, and the second chamber is a rectangular chamber attached to the bottom of the 96-well plate, wherein the second chamber is in electrochemical communication with each well in the 96-well plate by means of a porous membrane that separates each well from the rectangular chamber.

39. The method of claim 38, wherein the electrochemical cell is configured to provide a first electrode to two or more wells of the 96-well plate, and a single second electrode to the rectangular chamber, such that two or more samples of a biochemical composition may be reduced in parallel.

40. The method of claim 39, wherein the two or more samples comprise different biochemical compositions.

41. The method of any one of claims 30 to 40, wherein the first chamber contains the magnetic stir bar configured to agitate the one or more biochemical compositions.

42. The method of any one of claims 30 to 41, wherein the porous membrane is a cellulose membrane.

43. The method of any one of claims 30 to 42, wherein the porous membrane comprises a pore size small enough to retain the biochemical composition.

44. The method of claim 43, wherein the biochemical composition is a protein of molecular weight about 150 kDa.

45. The method of claim 43, wherein the biochemical composition is a protein of molecular weight about 50 kDa.

46. The method of any one of claims 30 to 45, wherein the first electrode is a negative electrode (or cathode).

47. The method of any one of claims 30 to 46, wherein the first electrode is a working electrode.

48. The method of claim 46 or claim 47, wherein the negative electrode comprises platinum.

49. The method of any one of claims 30 to 48, wherein the first electrode does not comprise titanium.

50. The method of claim 49, wherein the working electrode is connected to a direct current power supply.

51. The method of any one of claims 30 to 50, wherein the second electrode is a positive electrode (or anode).

52. The method of any one of claims 30 to 51, wherein the second electrode is a counter electrode.

53. The method of claim 51, wherein the positive electrode comprises carbon.

54. The method of any one of claims 30 to 53, wherein the biochemical composition is a protein.

55. The method of claim 54, wherein the protein is an antibody or an antibody fragment.

56. The method of any one of claims 30 to 55, wherein the first chamber and the second chamber comprise a buffer.

57. The method of claim 56, wherein the buffer comprises PBS.

58. The method of claim 56, wherein the buffer comprises glucose and EDTA.

59. The method of claim 55, wherein the first electrode and second electrode are submerged in the buffer.

60. The method of any one of claims 30 to 59, wherein the voltage or a process time are adjusted to control the extent of the reduction of the disulfide bonds, sulfur-selenium bonds, or diselenide bonds of the biochemical composition.

61. The method of any one of claims 30 to 60, further comprising conjugating a drug molecule, a detection agent, an imaging agent, a peptide, a protein, or an oligonucleotide to one or more sulfydryl groups derived from the disulfide bonds in a conjugation step.

62. The method of claim 61, wherein the conjugation step is performed in a buffer previously used for the reduction step without changing the buffer.
